(19) United States
(12) Patent Application Publication
(10) Pub. No.: US 2023/0270592 A1
(43) Pub. Date: Aug. 31, 2023

(54) A DEVICE FOR PROTECTING THE FACE OF A USER, IN PARTICULAR FOR THE EYES OF A USER

(71) Applicant: Guido PASQUANTONIO, Roma (RM) (IT)

(72) Inventor: Guido PASQUANTONIO, Roma (RM) (IT)

(21) Appl. No.: 18/008,119

(22) PCT Filed: Jun. 1, 2021
(86) PCT No.: PCT/IB2021/054780

§ 371 Date: Dec. 2, 2022

(30) Foreign Application Priority Data
Jun. 5, 2020 (IT) 202020000003178

(51) Int. Cl. A61F 9/02

(52) U.S. Cl. CPC A61F 9/028

(57) ABSTRACT

Described is a protective device for the face of a user, in particular for the eyes of a user, including a transparent visor designed to allow the user to see through the protective device and a supporting frame designed to allow the fixing of the protective device to the face of a user. The protective device has at least one opening designed to allow a passage of air from the inside to the outside and vice versa of an operating space defined, in use, by the protective device and by the face of the user. The protective device comprises a filtering device configured for filtering the air passing through said opening.

### TECHNICAL FIELD

This invention relates to a device for protecting the face of a user, in particular for the eyes of a user.

The invention relates to the technical field of personal protective equipment for the eyes of a user, in particular in the sector of goggles and eye masks.

### BACKGROUND ART

These devices are of significant importance for protecting operators in various industrial sectors.

In fact, an operator is subject to various types of risks that can put his/her eyes at risk.

For this reason, various personal protective devices have been developed, designed to protect the eyes of an operator from chippings, hot or caustic or corrosive materials, radiation, which may lead to mechanical, optical and thermal lesions.

In the medical field, for example, these devices must allow the user to operate in contaminating environments, guaranteeing that they are isolated from any infectious agents, for example present in the air in the form of droplets.

These devices may be equipped with different types of optical filters, for example for welding, ultraviolet rays, infrared rays and solar protection in such a way as to limit damage and disturbances correlated with them. Disadvantageously, such devices tend to mist up during use, for example due to the user's perspiration, limiting the vision of the user who is therefore forced to remove the device for cleaning.

Very disadvantageously, in situations in which the user operates in a potentially contaminated environment, the user is forced to move to a safe environment for cleaning the device.

Some more recent devices have aeration openings designed to allow a recirculation of the air in such a way as to limit the occurrence of misting up.

Disadvantageously, the aeration openings of these devices generate impediments and limit the field of vision reducing the comfort for the user, for example in the use of optical instruments such as microscopes.

Disadvantageously, the dust and the contaminants present in the air pass through these openings and settle inside the device, thus reducing the visibility.

The contaminating agents, for example of viral or bacterial origin, pass through the above-mentioned openings, subjecting the user's health to risks of various kinds.

In this context, the aim is to provide a protective device for the face of a user, in particular for the eyes of a user which overcomes the above-mentioned drawbacks of the prior art.

### AIM OF THE INVENTION

In particular, the aim of the invention is to provide a device for protecting the face of a user, in particular for the eyes of a user, which guarantees a high level of safety for the user during a plurality of operating conditions. This aim is achieved by a device for protecting the face of a user, in particular for the eyes of a user, comprising the technical features described in one or more of the appended claims.

The dependent claims correspond to possible embodiments of the invention.

### BRIEF DESCRIPTION OF THE DRAWINGS

Further features and advantages of the invention are more apparent in the non-limiting description which follows of a preferred embodiment of a device for protecting the face of a user, in particular for the eyes of a user, illustrated in the accompanying drawings, in which:

FIG. 1 is a side view of a possible embodiment of a device for protecting the face of a user, in particular for the eyes of a user, according to the invention;

FIGS. 2 to 4 are perspective views of some possible embodiments of a portion of the device of FIG. 1;

FIGS. 5 to 6 are perspective views of some further possible embodiments of a portion of the device for protecting the face of a user, in particular for the eyes of a user, according to the invention;

FIG. 7 is a side view of a possible embodiment of a device for protecting the face of a user, in particular for the eyes of a user, according to the invention;

FIGS. 8 and 9 are details of an embodiment of a protective device according to the invention.

### DETAILED DESCRIPTION OF PREFERRED EMBODIMENTS OF THE INVENTION

With reference to the accompanying drawings, the numeral 1 denotes in its entirety a device for protecting the face of a user, in particular for the eyes of a user, which will hereinafter be referred to, for simplicity of description, as protective device 1.

The protective device 1 comprises a visor 2 configured to protect the eyes of a user during a plurality of operating conditions.

The visor 2 is made of transparent material in such a way as to allow the user to see clearly and sharply through the protective device 1 during use of the protective device 1.

Advantageously, the visor 2 may have a pair of recesses for positioning, in a position close to the eyes of a user, viewing and/or viewing amplification instruments.

In this way, the user can easily use these instruments without the need to remove the protective device 1.

Preferably, the visor 2 is made of polymeric material and/or by a plastic moulding process.

Advantageously, the visor 2 may have one or more anti-glare portions and/or with increased opacity in such a way as to improve the comfort of the user.

The protective device 1 comprises a frame 3 for supporting the visor 2 designed to allow the fixing to the face of a user U.

Advantageously, the visor 2 and the supporting frame 3 may be made in a single monolithic piece of transparent material, guaranteeing that the view of the user U is substantially free of obstacles.

Preferably, the supporting frame 3 comprises one or more supporting portions designed to be positioned, in use, resting on the face of the user U.

Advantageously, the supporting portions may be made of flexible polymeric material, preferably silicone rubber, in such a way as to guarantee a high level of comfort during use.

The protective device 1 may be shaped in such a way as to define, in use, a sealed coupling with the face of the user U.

The supporting frame 3 may comprise generic fixing means (not illustrated in the accompanying drawings), for example one or more elasticized bands and/or supporting sidepieces, designed to fix the device 1 to the head of the user U.

Advantageously, the protective device 1 has at least one opening 4 designed to allow a passage of air from the inside to the outside and vice versa of an operating space “V” defined, in use, by the protective device and by the face of the user U.

In an embodiment illustrated, the opening 4 is made in the supporting frame 3.

According to an embodiment not illustrated, the opening 4 is made in the visor 2.

In other words, the protective device 1 and the face of the user, in use, delimit an operating space “V” in which the air can circulate through the opening 4.

In this way, the protective device 1 allows, for example, an outflow of the moist air present inside the operating space, limiting, preferably preventing, the misting up of the visor 2.

The opening 4 may have a substantially circular transversal cross section. More specifically, the diameter of the above-mentioned circular section may be between 2 mm and 70 mm, preferably 30 mm.

Advantageously, the protective device 1 comprises a filtering device 5 configured for filtering the air passing through the opening 4.

The filtering device 5, filtering the air entering the operating space “V”, prevents any contaminating agents, for example of viral or bacterial origin in the form of droplets and/or in other forms, and/or powders of various kinds, from coming into contact with the portion of the face of the user U covered by the protective device 1.

In this way, the filtering device 5 increases the safety of the user preventing the user from coming into contact with contaminating agents. The filtering device 5 prevents the powder, entering the operating space “V”, from being deposited on the transparent surfaces so as to limit the viewing of the user.

Advantageously, the filtering device 5 can be made separately from the protective device 1 and then applied to eye protection devices of various types, guaranteeing high application flexibility.

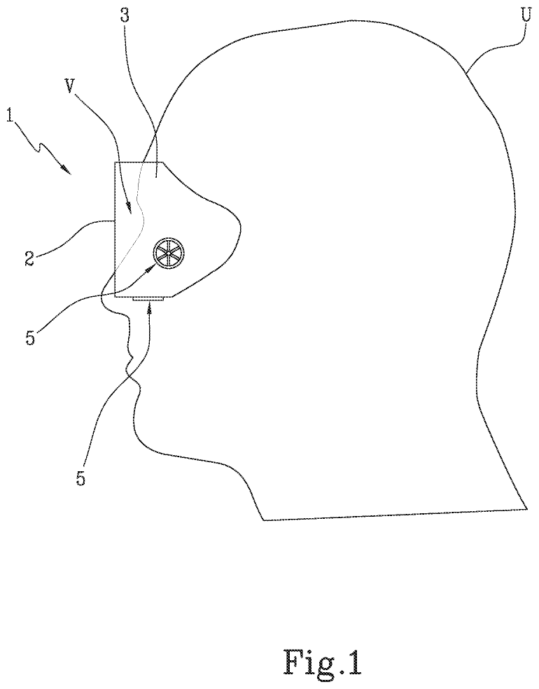

According to a possible embodiment and as illustrated in FIG. 1, the protective device 1 may have a plurality of openings and comprise a plurality of respective filtering devices 5 guaranteeing an effective recirculation of air through the operating space “V”.

Advantageously, the filtering device 5 is connected or connectable to the supporting frame 3 in such a way as not to generate interference with the vision of the user U.

For example, the filtering device 5 may be connected to the supporting frame 3 and be positioned, in use, substantially close to the head of the user.

In a preferred embodiment, the filtering device 5 comprises a filtering element 6 designed to filter the air which passes through the opening 4.

In one embodiment, the filtering device consists of the filtering element 6.

Preferably, the filtering element 6 is a “class 1” (FFP1), “class 2” (FFP2) or “class 3” (FFP3) filter.

The filtering element 6 may comprise one or more filtering layers made, for example, of polypropylene and/or polycarbonate, antibacterial cotton and/or fabric of various types, preferably non-woven fabric.

More specifically, the filtering element 6 may comprise a plurality of filtering layers superposed and connected to each other. Preferably, these layers can be connected by ultrasonic sealing in such a way as to guarantee adherence between the layers and an effective filtering action even under conditions of high humidity.

Advantageously, one or more filtering layers can be charged electrostatically in such a way as to increase the effectiveness of the filtering action.

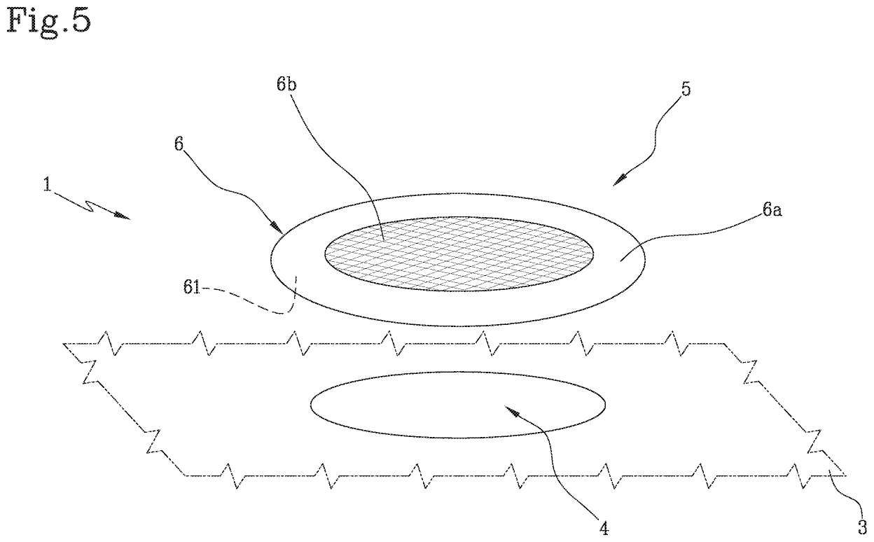

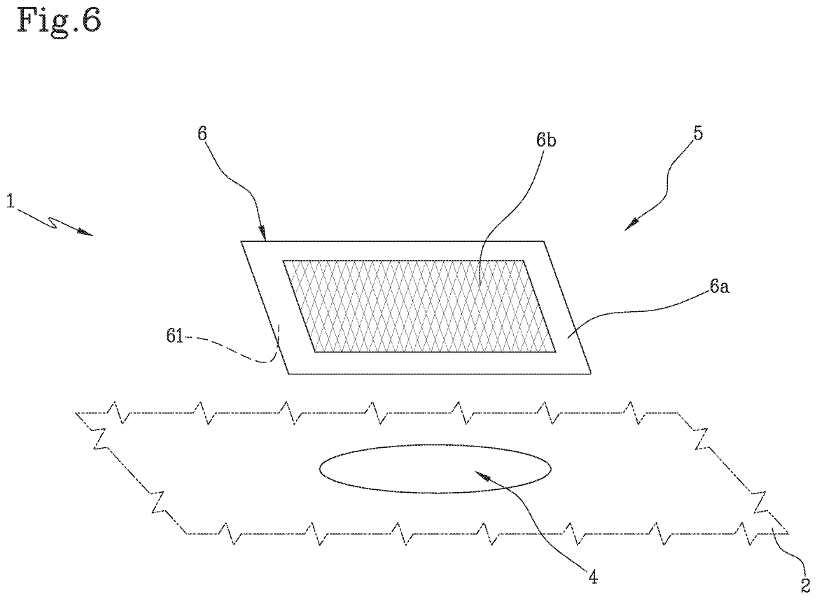

According to a possible embodiment and as illustrated for example in FIGS. 5-6, the filtering element 6 is connected, or connectable, to the visor 2 and/or the supporting frame 3 facing the opening 4 by gluing. The filtering element 6 comprises a portion 6a, preferably a perimeter portion, designed to define the connection with the visor 2 and/or the supporting frame 3 and a portion 6b, preferably central, designed to filter the air which passes through the opening 4.

In a preferred embodiment, the perimeter portion 6a surrounds at least partly the central portion 6b.

In one embodiment, the perimeter portion 6a comprises an adhesive layer 61 on at least one surface, preferably on both surfaces, for connecting, preferably in a reversible fashion, the filtering element 6 to the visor 2 and/or to the supporting frame 3.

The adhesive layer 61 allows the filtering element 6 to be applied at the opening 4.

The filtering element 6 may be applied inside the space V and/or outside of it at the opening 4.

The invention makes it possible to protect at least the eyes of the user U, preferably using filtering fabrics normally used for protecting the airways. Advantageously, the adhesive layer 61 allows the filter to be applied also on aeration valves of known type which are provided in the protective device 1.

The filtering element 6 preferably has a substantially circular shape (as illustrated in FIG. 5).

In other embodiments, the filtering element 6 may have different shapes, for example quadrangular or rectangular (as illustrated in FIG. 6).

According to an embodiment, the filtering device 5 projects from a surface of the visor 2 and/or of the supporting frame 3 outside the operating space “V” in such a way as to limit the overall dimensions inside the operating space “V”.

According to an embodiment, the filtering device 5 is at least partly inside the operating space “V”.

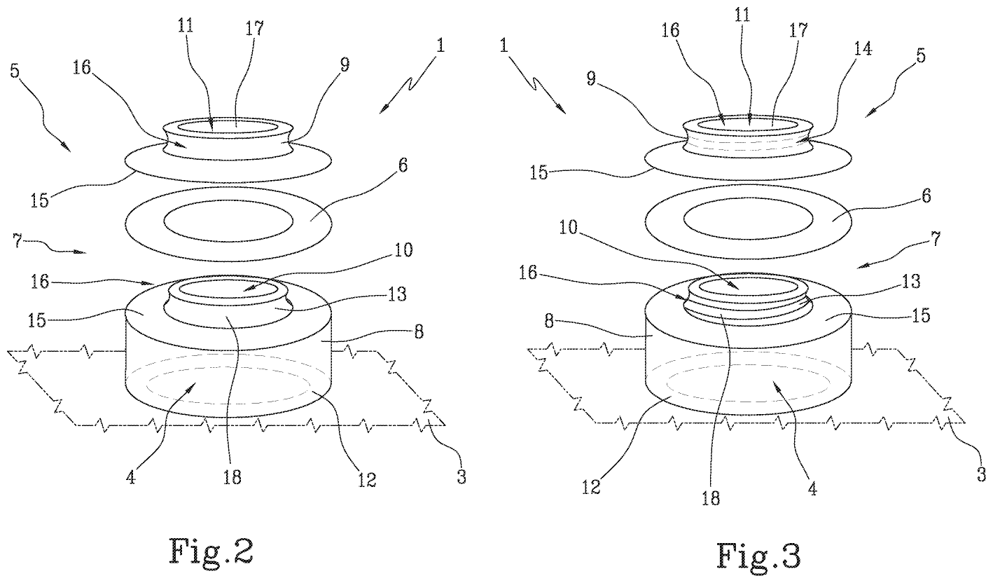

In an embodiment illustrated by way of example in FIGS. 2 to 4, the filtering device 5 comprises a body 7 preferably substantially tubular or annular in shape.

In one embodiment, the element 7 is in fluid communication with the opening 4 and the filtering element 6 is associated with the body 7 for filtering the air passing through the opening 4.

Preferably, the element 7 may comprise a supporting element 8 and a locking element 9 having respective openings 10, 11 in fluid communication with the opening 4.

Preferably, the supporting element 8 has a substantially annular or tubular shape.

More specifically, the supporting element 8 is connected or connectable to the protective device 1.

According to some possible embodiments, the supporting element 8 is made as one piece, preferably using a plastic moulding process, with the frame 3 and/or the visor 2.

According to possible embodiments, the supporting element 8 is connected, for example by gluing, to the frame 3 and/or the visor 2.

In an embodiment not illustrated, the filtering device comprises accessories such as micro-fans or similar accessories for maximising ventilation. Preferably, the accessories are installed on the supporting element 8 or the supporting element 8 comprises the accessories.

According to an embodiment, the locking element 9 is reversibly connectable to the supporting element 8 in such a way as to lock the filtering element 6 between the locking element 9 and the supporting element 8.

More specifically, the supporting element 8 preferably comprises a fixing portion 12 connected or connectable to the protective device 1 and a locking portion 13 connectable to a respective locking portion 14 of the locking element 9.

Advantageously, the locking portion 13 may be thinner than the fixing portion 12. The locking element 9 and the locking portion 13 may be shaped to match to form an effective coupling which has limited dimensions and a high aesthetic appeal.

In one embodiment, at least one filtering layer may comprise a perimeter portion with increased thickness designed to facilitate the locking of the filtering element 6 in one or more operating conditions.

As illustrated in FIGS. 2 to 4, the supporting element 8 and the locking element 9 have respective locking surfaces 15 which can be activated on the filtering element 6 to define a locking thereof.

More specifically, in use, the filtering element 6 is at least partly interposed between the locking surfaces 15.

In other words, the coupling between the locking element 9 and the supporting element 8 activates the locking surfaces 15 on the filtering element 6 to lock it firmly in contact with the opening 4. The protective device 1 may comprise coupling means 16 designed to reversibly connect the locking element 9 to the supporting element 8.

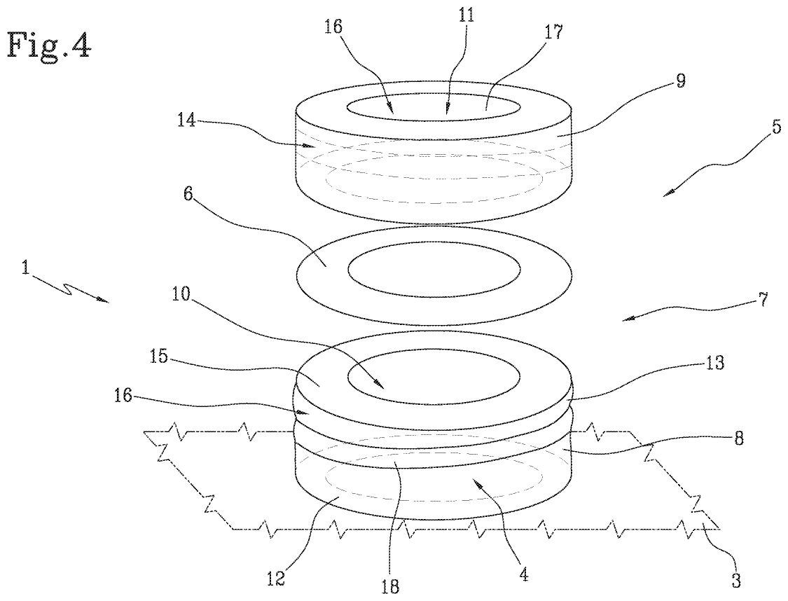

Preferably, the coupling means 16 comprise snap-on couplings (as illustrated in FIG. 2), bayonet couplings, couplings of the lead nut and screw type (as illustrated in FIGS. 3, 4) and/or magnetic couplings.

More specifically, the coupling means 16 may comprise an inner surface 17 of the locking element 9 and an outer surface 18 of the supporting element 8.

According to a possible embodiment and as illustrated in FIGS. 3 and 4, the inner surface 17 of the locking element 9 and the outer surface 18 of the supporting element 8 are at least partly threaded, making possible a lead nut and screw type coupling between the locking element 9 and the supporting element 8.

In other words, the locking portion 13 of the supporting element 8 may be externally threaded to define a coupling with the inner surface 17 of the locking element 9.

More specifically, the locking portion 13 is threaded and may be thinner than the fixing portion 12 (as illustrated in FIG. 3) or it may have the same thickness as the fixing portion 12 and, therefore, the threaded portion may extend on at least one portion of the lateral surface of the supporting element 8.

Preferably, the filtering device 5 may comprise sealing means (not illustrated in the accompanying drawings), for example an elastomeric O-ring, designed to be interposed between the supporting element 8 and the locking element 9 in such a way as to guarantee a high seal of the protective device 1 to prevent the entrance into the operating space “V” of contaminants or the like.

The protective device 1 may comprise at least one connecting element (not illustrated in the accompanying drawings) designed to connect the locking element 9 to one or more between the supporting element 8, the visor 2 and/or the supporting frame 3.

In accordance with a preferred, non-limiting embodiment, the connecting element may have the shape of a cord, a chain or the like and be made of polymeric material, metal and/or fabric.

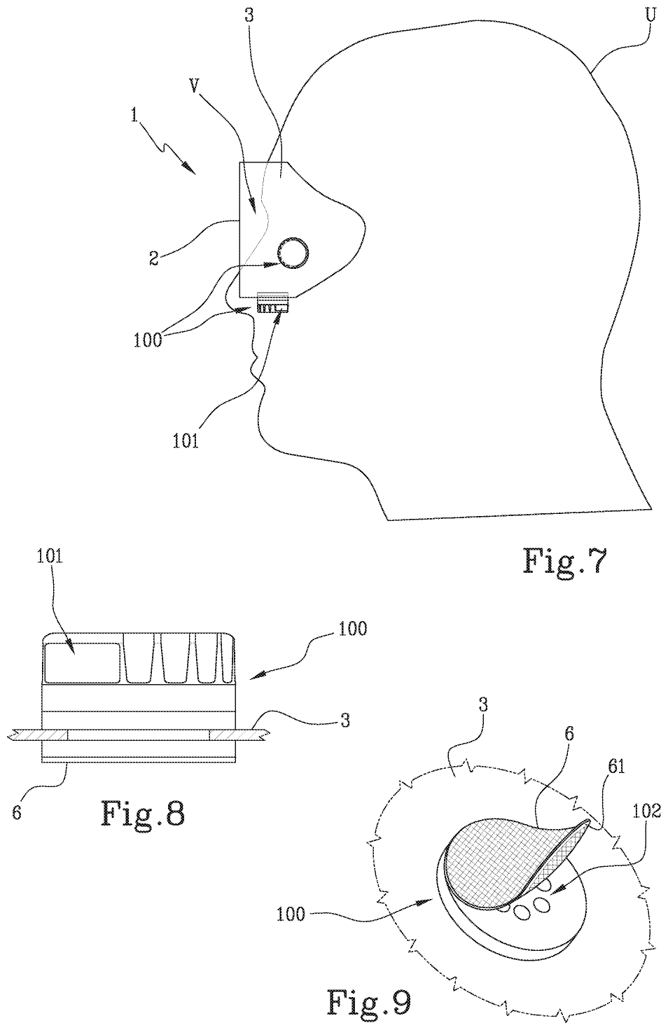

In one embodiment, illustrated for example in FIGS. 7 to 9, the protective device 1 comprises a ventilation valve 100 inserted in the opening 4 and a filtering element 6 applied to the valve 100. In general, the valve 100 has an anti-misting up function.

The valve 100, of substantially known type and described only insofar as necessary for understanding the invention, is adjustable between an open configuration, wherein it allows a passage of air from the outside to the inside of the space V and vice versa, and a closed configuration.

The valve 100 has an inlet 101 and an outlet 102 which are in fluid communication when the valve 100 is in the open configuration. For practicality, it is assumed that the outlet 102 faces the space V and the inlet 101 faces the outside of it.

In one embodiment, the filtering element 6 is applied, preferably by means of the adhesive layer 61, to the valve 100 at the outlet 102.

In one embodiment, the filtering element 6 is applied, preferably by means of the adhesive layer 61, to the valve 100 at the inlet 101.

In one embodiment, the filtering element 6 is attached to the frame 3 and/or to the visor 2 at the valve 100 preferably by the adhesive layer 61.

It should be noted, therefore, that the protective device achieves the preset aims as it is able to guarantee a high level of safety for the user during a plurality of operating conditions thanks to the presence of a filtering device configured for filtering the air passing through the aeration openings.

Advantageously, the protective device guarantees a high level of comfort for the user preventing the misting up during use and, at the same time, increasing the safety of the user, preventing the user from coming into contact with any contaminating agents present in the air, for example droplets or dust.

Advantageously, the positioning of the filtering device does not obstruct the vision of the user, guaranteeing high comfort and simplifying the activities.

1. A device for protecting the face of a user, in particular for the eyes of the user, comprising:
a transparent visor;
a frame for supporting said visor to allow said protective device to be fixed to the face of the user;
said protective device having at least one opening for allowing a passage of air from the inside to the outside and vice versa of an operating space defined, in use, by the protective device and by the face of the user; said protective device comprising at least one filtering device configured to filter the air passing through said opening, said filtering device comprising a filtering element connected or connectable to said visor and/or to said supporting frame and facing said opening for filtering the air passing through said opening.

2. The protective device according to claim 1, wherein said filtering element comprises an adhesive layer for joining with said visor and/or to said supporting frame at said opening.

3. The protective device according to claim 2, wherein said filtering element comprises a perimeter portion for connecting with said visor and/or to said supporting frame and a central portion, said perimeter portion comprising said adhesive layer.

4. The protective device according to claim 1, wherein said filtering element is applied inside said operating space.

5. The protective device according to claim 1, comprising a ventilation valve inserted in said opening, said filtering element being applied to said ventilation valve and being joined to said visor and/or to said supporting frame using said valve.

6. The protective device according to claim 5, wherein said valve has an inlet and an outlet conventionally facing said operating space, said filtering element being applied at said outlet.

7. The protective device according to claim 1, wherein said filtering element is applied to said visor and/or to said supporting frame at said opening.

8. The protective device according to claim 1, wherein said filtering device comprises a body preferably having a substantially tubular or annular shape in fluid communication with said opening, said filtering element being associated with said body and facing said opening.

9. The protective device according to claim 8, wherein said body comprises a supporting element and a locking element having respective openings in fluid communication with said opening, said supporting element being connected or connectable to said visor and/or to said supporting frame and said locking element being reversibly connectable to said supporting element, said filtering element being positioned and locked between said locking element and said supporting element.

10. The protective device according to claim 9, comprising coupling means designed to reversibly connect said locking element to said supporting element, said coupling means preferably comprising snap-on couplings, bayonet couplings, lead nut and screw type couplings and/or magnetic couplings.

11. The protective device according to claim 9, wherein said supporting element is made as one piece with said supporting frame and/or with said visor.

12. A filter comprising a filtering element comprising a perimeter portion and a central portion surrounded at least partly by the perimeter portion, said filtering element comprising an adhesive layer positioned at least partly on the perimeter portion for joining the filter to a support.

13. The filter according to claim 12, wherein said central portion comprises one or more filtering layers made, for example, of polypropylene and/or polycarbonate microfibre, antibacterial cotton and/or fabric of various types, preferably non-woven fabric, said filtering layers being preferably superposed and connected to each other.
